(19) United States
(12) Patent Application Publication
(10) Pub. No.: US 2026/0345985 A1
(43) Pub. Date: Nov. 5, 2026

(54) DETACHABLE INTRAVENOUS LINE CONNECTOR

(71) Applicant: CareFusion 303, Inc., San Diego, CA (US)

(72) Inventor: Adel SHAMS, Verona, NJ (US)

(21) Appl. No.: 19/197,779

(22) Filed: May 2, 2025

(51) Int. Cl. A61M 39/10; A61M 39/12; A61M 39/26

(52) U.S. Cl. CPC A61M 39/1011; A61M 39/12; A61M 39/26; A61M 2039/1061; A61M 2039/261; A61M 2039/263; A61M 2039/268

(57) ABSTRACT

An anti-dislodgement intravenous (IV) line connector includes a poppet valve positioned within a housing. The poppet valve has a fluid chamber configured to receive fluid from an IV set and a plug that is slidable relative to the fluid chamber to control fluid flow through the poppet valve. The housing has a snap-fit rim that is coupled to the poppet valve and a skirt configured to receive a fluid reception device. When the connector is coupled to the fluid reception device, the fluid reception device distally advances the plug, which fluidly connects the fluid chamber of the poppet valve to the fluid reception device. When a sufficient force is applied to the IV set, the snap-fit rim separates from the poppet valve to decouple the housing from the connector and fluidly close the IV line before the IV set is dislodged from the patient.

### TECHNICAL FIELD

The present invention relates generally to intravenous (IV) line connectors and, in particular, to a detachable IV line connector assembly that limits IV line dislodgements.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag.

In some applications, IV catheters may become dislodged from the patient when the tubing and/or fittings are subject to forces greater than what they are designed to withstand. Disconnection of IV catheters from the patient can cause pain to the patient. In some cases, abrupt dislodgements can cause infections. Additionally, when an IV catheter is dislodged (or when other parts of the IV set are disconnected), fluid can leak out of the IV set, which is inconvenient for caregivers who have to clean up the spill and raises the cost of treatment by wasting medical fluid.

### SUMMARY

An intravenous (IV) line connector assembly that limits IV catheter and IV set dislodgements can be desirable to mitigate discomfort and pain inflicted on patients receiving infusion treatment and reduce the occurrence of fluid spilling out of IV sets. Accordingly, some embodiments of the present disclosure are directed to an anti-dislodgement connector assembly configured to connect to an intravenous (IV) set and comprising a reception connector and a delivery connector, wherein the reception connector comprises a female luer and a deformable valve disposed within the female luer, and the delivery connector comprises: a housing comprising a proximal skirt and a snap-fit rim opposite the skirt; an inlet port coupled to the snap-fit rim and configured to transport fluid from the IV set to the delivery connector; a poppet valve coupled to the inlet port, proximally abutting the snap-fit rim, and comprising (a) a fluid chamber configured to receive the fluid from the inlet port and comprising a proximal outlet and (b) a plug comprising (i) a distal post positioned within the fluid chamber and coupled to the inlet port by a biasing member, (ii) a proximal post, and (iii) a sealing region positioned between the distal post and the proximal post and movable relative to the proximal outlet, wherein the proximal skirt and the poppet valve form a male luer configured to couple with the female luer of the reception connector, the poppet valve comprising a taper of the male luer, wherein, when the male luer is coupled with the female luer, (a) the poppet valve engages against the deformable valve such that the plug is distally displaced, thereby compressing the biasing member and forming a fluid passage between the sealing region and the proximal outlet and (b) the deformable valve deforms against the proximal post to fluidly couple the fluid chamber to the proximal outlet, wherein, when a force is applied to the IV set, the snap-fit rim slides away from the inlet port and over the poppet valve to decouple the housing from the inlet port and the poppet valve, and the biasing member proximally advances the sealing region into the proximal outlet to seal the fluid chamber.

In some embodiments, the biasing member comprises a spring.

In some embodiments, when the male luer is not coupled with the female luer, there is a gap between the inlet port and the distal post, and the biasing member extends from the distal post and over the gap to contact the inlet port. Optionally, the distal post is at least partially positioned within an interior of the biasing member.

In some embodiments, the force is at least about 4 pounds.

In some embodiments, when the force is applied to the IV set, the housing decouples from the inlet port and the poppet valve before the IV set is dislodged from a patient.

Some embodiments of the present disclosure are directed to an anti-dislodgement connector configured to connect to an intravenous (IV) set, the connector comprising: a housing comprising a snap-fit rim and a proximal skirt configured to couple to a fluid reception device of the IV set; an inlet port coupled to the snap-fit rim and configured to receive fluid from the IV set; and a poppet valve coupled to the inlet port, proximally abutting the snap-fit rim, and comprising a fluid chamber and a plug, the fluid chamber configured to receive the fluid from the inlet port and connected to a surrounding environment by a proximal outlet, and the a plug comprising a distal post, positioned within the fluid chamber and coupled to the inlet port by a biasing member, and a proximal sealing region that is movable relative to the proximal outlet, wherein the proximal skirt and the poppet valve form a male luer and, when the male luer is coupled to the fluid reception device, the plug is distally advanced to compress the biasing member, distally displace the sealing region and fluidly couple the fluid chamber to the proximal outlet, and wherein, when a force is applied to the IV set, the snap-fit rim slides away from the inlet port and over the poppet valve to decouple the housing from the inlet port and the poppet valve, and the biasing member proximally advances the sealing region into the proximal outlet to seal the fluid chamber.

In some embodiments, the snap-fit rim is positioned at a distal end of the housing and comprises a lip that protrudes radially inwards.

In some embodiments, the inlet port is welded to the poppet valve.

In some embodiments, a distal end of the poppet valve proximally abuts the snap-fit rim and comprises a tapered edge, the snap-fit rim being configured to slide up the tapered edge when the force is applied to the IV set.

In some embodiments, the distal post is at least partially positioned within an interior of the biasing member.

In some embodiments, the plug further comprises a proximal post protruding proximally from the sealing region, the proximal post being configured to engage against the fluid reception device when the connector is coupled to the fluid reception device.

In some embodiments, the proximal outlet and the sealing region are both proximally tapered radially inward, and, when the male luer is not coupled to the fluid reception device, the sealing region fits within the proximal outlet such that the sealing region seals the proximal outlet.

In some embodiments, the force is at least about 4 pounds.

Other embodiments of the present disclosure are directed to an anti-dislodgement connector configured to connect to an intravenous (IV) set, the connector comprising: a housing with a snap-fit rim; an inlet port coupled to the snap-fit rim and configured to receive fluid from the IV set; and a poppet valve positioned proximal to the snap-fit rim and within the housing, the poppet valve comprising a fluid chamber and a plug, the fluid chamber configured to receive the fluid from the inlet port and connected to a surrounding environment by a proximal outlet, and the plug coupled to the inlet port by a biasing member and movable relative to the proximal outlet, wherein, when the connector is coupled to a fluid reception device of the IV set, the biasing member is compressed and the plug is distally displaced to expose the proximal outlet, thereby fluidly connecting the fluid chamber to the proximal outlet, and wherein, when a force is applied to the IV set, the snap-fit rim slides away from the inlet port and over the poppet valve to decouple the housing from the inlet port and the poppet valve, and the biasing member proximally advances the plug into the proximal outlet to seal the fluid chamber.

In some embodiments, a distal end of the poppet valve proximally abuts the snap-fit rim and comprises a tapered edge, the snap-fit rim being configured to slide up the tapered edge when the force is applied to the IV set.

In some embodiments, the plug further comprises a post protruding proximally from the plug, the post being configured to engage the fluid reception device to form a fluid path from the fluid chamber and the proximal outlet to the fluid reception device.

In some embodiments, the proximal outlet and the plug are both proximally tapered radially inward, and, when the connector is not coupled to the fluid reception device, the plug fits within the proximal outlet such that the plug seals the proximal outlet.

In some embodiments, the force is at least about 4 pounds.

In some embodiments, a proximal portion of the housing and the poppet valve form a male luer at a proximal portion of the connector, with the proximal portion of the housing forming a skirt and the poppet valve forming a taper.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an intravenous (IV) set coupled to a patient, in accordance with aspects of the present disclosure.

FIG. 2 illustrates a side view of an anti-dislodgement connector assembly before the reception connector and the delivery connector are coupled together, in accordance with aspects of the present disclosure.

FIG. 3 illustrates a cross-sectional view of an anti-dislodgement connector assembly before the reception connector and the delivery connector are coupled together, in accordance with aspects of the present disclosure.

FIG. 4 illustrates a cross-sectional view of the anti-dislodgement connector assembly of FIG. 3 with the reception connector and the delivery connector coupled together, in accordance with aspects of the present disclosure.

FIG. 5 illustrates a cross-sectional view of an anti-dislodgement connector assembly of FIG. 3 after the reception connector and the delivery connector have been decoupled by an outside force, in accordance with aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

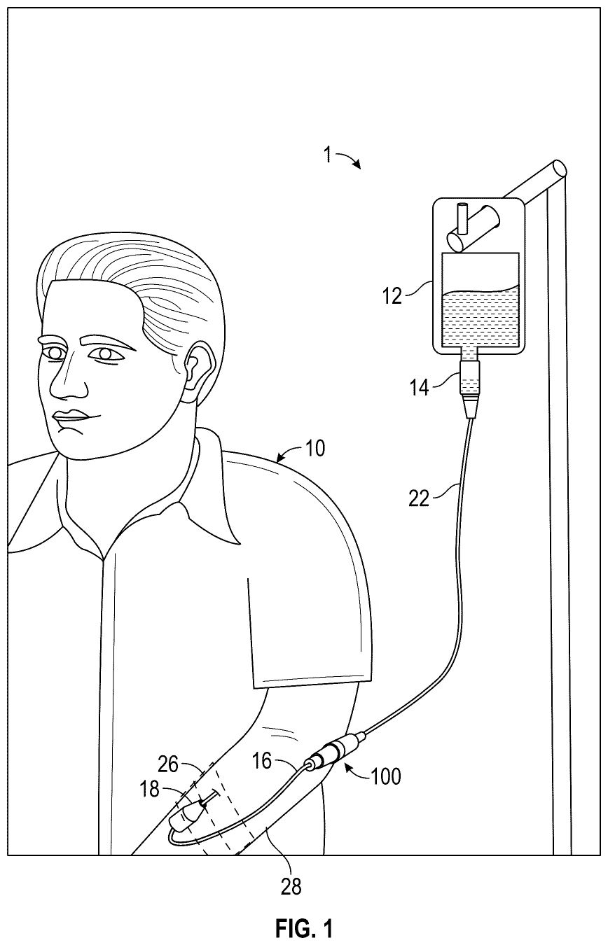

Referring now to the figures, FIG. 1 illustrates an IV set coupled to a patient, in accordance with aspects of the present disclosure. The IV set 1 includes a medical fluid bag 12, a drip chamber 14, a first tube 22, a fluid connector assembly 100, a second tube 16, a catheter 18, and tape 26. The tubing 22 extends between the drip chamber 14 and the fluid connector assembly 100. The tubing 16 extends between the fluid connector assembly 100 and the catheter 18, which is inserted in the arm 28 of the patient 10. In some embodiments, to resist unintended dislodgement or disconnection of the tubing 16 or the catheter 18 from the patient, the tape 26 is placed over the tubing 16 and the catheter 18, so that the tape 26 engages the tubing 16, the catheter 18, and the patient's arm 28.

The fluid connector assembly 100 is designed for use in medical applications, such as the IV set 1 as well as other IV medical fluid delivery applications using catheters, including peripheral intravenous catheters (PIVC), as non-limiting examples. In some embodiments, the fluid connector assembly 100 is a needle-free fluid connector. In other embodiments, the fluid connector assembly 100 is an anti-dislodgement connector assembly. Optionally, the fluid connector assembly 100 is both needle-free and designed to resist dislodgement of the IV set 1 (in particular, the catheter 18) from the patient 10.

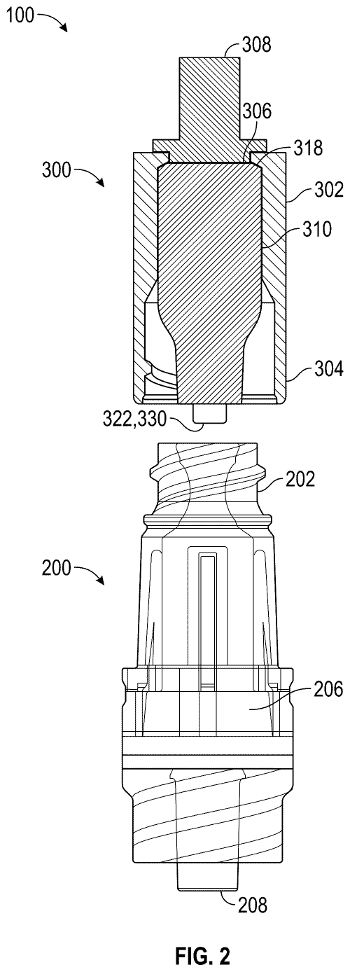

FIG. 2 illustrates a side view of an anti-dislodgement connector assembly before the reception connector and the delivery connector are coupled together, in accordance with aspects of the present disclosure. The anti-dislodgement connector assembly 100 can include two parts: the reception connector 200 and the delivery connector 300. In the embodiment shown, the anti-dislodgement connector assembly 100 is a needle-free fluid connector system, and both the reception connector 200 and the delivery connector 300 are needle-free fluid connectors.

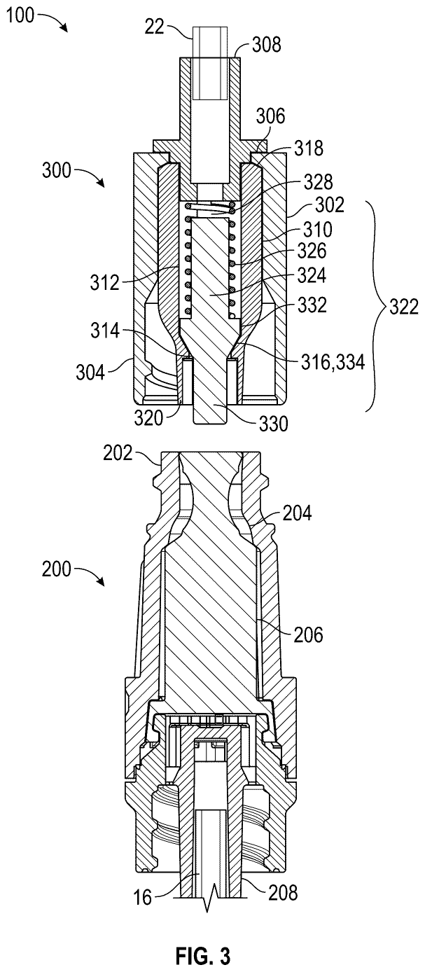

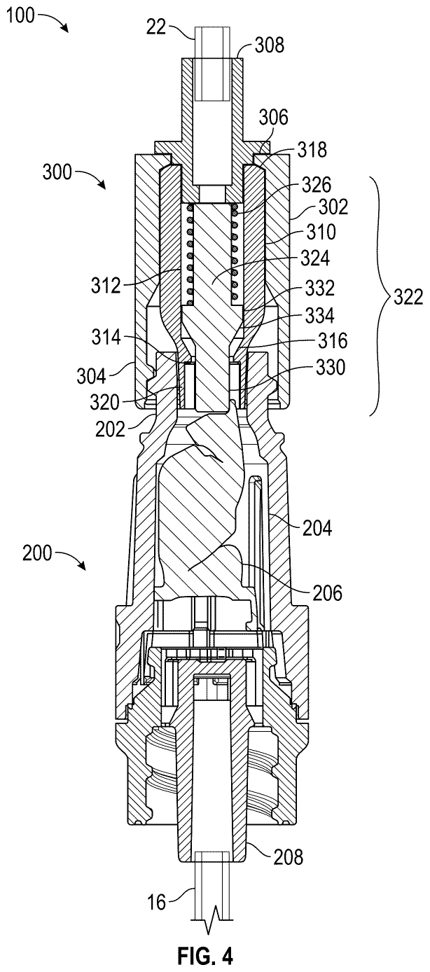

The reception connector 200 includes a luer 202 positioned at a distal portion of the reception connector 200. In some embodiments, the outer surface of the luer 202 (i.e., the outer surface of the distal region of the reception connector 200) comprises flanges or lugs. The reception connector 200 also has a deformable valve 206 positioned within a lumen (i.e., lumen 204, which is depicted in FIGS. 3-5). When the deformable valve 206 is in its default configuration (as shown in FIGS. 2, 3, and 5), the deformable valve 206 fills the luer 202, thereby sealing the luer 202 and preventing fluid flow into the lumen and through the reception connector 200. The deformable valve 206 can be compressed by the delivery connector 300 into a deformed configuration (as shown in FIG. 4). When the deformable valve 206 is in the deformed configuration, the luer 202 is not sealed, and fluid can enter the reception connector 200, flow through the lumen, and exit through the outlet 208.

The delivery connector 300 includes a housing 302 with a proximal skirt 304 that is couplable with the luer 202 of the reception connector 200. The proximal skirt 304 includes a hollow interior at a proximal portion of the housing 302. In some embodiments, the inner surface of the proximal skirt 304 (i.e., the surface of the hollow interior) comprises a threaded surface that is configured to receive the flanges or lugs of the luer 202. The distal portion of the housing 302 also has a snap-fit rim 306, which releasably couples the housing 302 to the other components of the delivery connector 300 and is discussed in greater detail below with respect to FIG. 3. An inlet port 308 is positioned at the distal portion of the delivery connector 300 and can be coupled to an IV set (such as IV set 1 via IV tube 22 in FIG. 1) to transport fluid into the delivery connector 300. The inlet port 308 is releasably coupled to the housing 302 via the snap-fit rim 306 and is attached to the poppet valve 310. The poppet valve 310 is positioned within the housing 302. When neither the poppet valve 310 nor the delivery connector 300 are coupled to the reception connector 200, the poppet valve 310 is in a sealed configuration (as shown in FIGS. 2, 3, and 5). In the sealed configuration, fluid does not exit the delivery connector 300. On the other hand, when the poppet valve 310 and/or the entire delivery connector 300 is coupled to the reception connector 200, the poppet valve 310 is in a flow configuration (as shown in FIG. 4), in which fluid can flow through the delivery connector 300.

When the reception connector 200 and the delivery connector 300 are coupled together (as shown in FIG. 4) and the assembly 100 is connected to an IV set (such as IV set 1 in FIG. 1), fluid from a medical fluid bag (e.g., medical fluid bag 12 in FIG. 1) enters the proximal end of the delivery connector 300 via the inlet port 308. Because the reception connector 200 and the delivery connector 300 are coupled together, the poppet valve 310 is in the flow configuration and the deformable valve 206 is in the deformed configuration. Consequently, the delivery connector 300 can deliver the fluid to the reception connector 200, and the fluid can flow through the lumen and exit the reception connector 200 via the outlet port 208 to be delivered to the patient. The structural components that create this flow path are discussed in greater detail below with respect to FIG. 3.

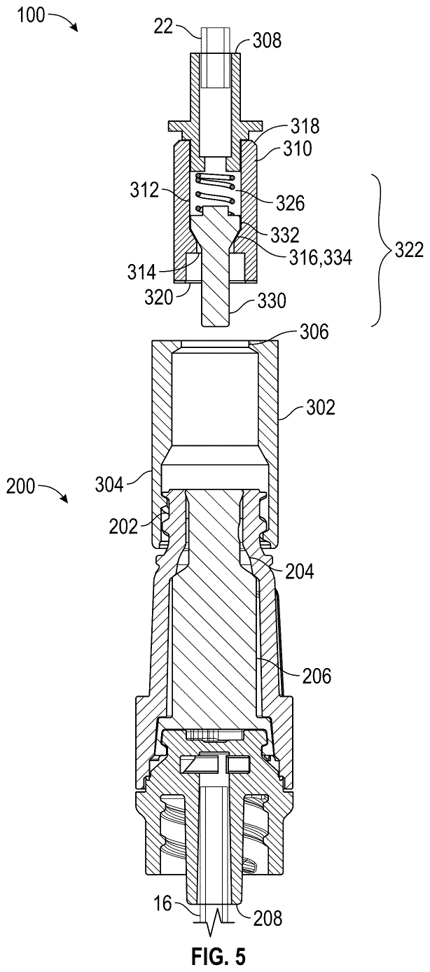

When a force that matches or exceeds a set threshold is applied to the IV set, the reception connector 200 and the delivery connector 300 separate or decouple. In some embodiments, the force is a linear force that can be applied in any direction relative to the reception connector 200 and the delivery connector 300. For example, the force can be axial to the assembly 100 (i.e., parallel to the flow path through the IV set) or transverse to the assembly 100. In other embodiments, the force is any force that biases the housing 302 away from the poppet valve 310(or that biases the poppet valve 310 away from the housing 302). In response to this force, the skirt 304 remains coupled with the luer 202, so the housing 302 separates from the remainder of the delivery connector 300 (i.e., the inlet port 308 and the poppet valve 310), as will be described in greater detail below with respect to FIG. 5. When the reception connector 200 and the delivery connector 300 decouple, the deformable valve 206 returns to the default configuration, and the poppet valve 310 returns to the sealed configuration. In some embodiments, the set threshold force is about three pounds. In other embodiments, the set threshold force is about four pounds. In yet other embodiments, the set threshold force is about five pounds. Accordingly, when a force that matches or exceeds three, four, or five pounds is applied to the IV set, the reception connector 200 and the delivery connector 300 will decouple. Optionally, the reception connector 200 and the delivery connector 300 will decouple before the IV set is removed from the patient (e.g., with respect to FIG. 1, before the catheter 18 is pulled out of the arm 28 of the patient 10). For example, the reception connector 200 and the delivery connector 300 are configured to decouple in response to a four pound force, and a four and a half pound force is strong enough to dislodge the IV set from the patient. On the other hand, when a force below the set threshold is applied to the IV set, the connector assembly 100 remains coupled, and fluid continues to flow through the connector assembly 100.

FIG. 3 illustrates a cross-sectional view of an anti-dislodgement connector assembly before the reception connector and the delivery connector are coupled together, in accordance with aspects of the present disclosure. As shown, a distal portion of the reception connector 200 includes a luer 202, and a proximal portion of the reception connector 200 includes an outlet 208. The luer 202 is configured to couple with a skirt 304 of the delivery connector 300, and the outlet 208 is configured to couple with an IV line 16 (which corresponds to the IV tube 16 of the IV set 1 in FIG. 1). As shown by FIG. 3, the luer 202 can be sealed by the deformable valve 206 when the deformable valve 206 is fully expanded and presses radially outward against the luer 202. This can be referred to as the default configuration of the deformable valve 206. Moreover, the deformable valve 206 is positioned within the lumen 204 and, in the default configuration, seals the outlet 208. Accordingly, in the default configuration depicted in FIG. 3, the deformable valve 206 fluidly disconnects the luer 202 from the outlet 208.

The delivery connector 300 has a housing 302, an inlet port 308, and a poppet valve 310. The proximal portion of the housing 302 includes a skirt 304 that is couplable with the luer 202 of the reception connector 200. In the embodiment shown in the present application, the skirt 304 and the proximal portion of the poppet valve 310 form a male luer that can couple with the female luer 202.

The distal portion of the housing 302, opposite the skirt 304, includes a snap-fit rim 306. The housing 302 is generally hollow, and the snap-fit rim 306 comprises a lip formed around an opening at the distal portion of the housing 302. The lip protrudes radially inwards from an outer surface of the housing 302 and then tapers radially outward to align with an inner surface of the housing 302. As depicted in FIG. 3, the snap-fit rim 306 and the distal portion of the housing 302 snap fit or friction fit around the distal portion of the poppet valve 310. The poppet valve 310 has a tapered distal edge 318 that proximally abuts tapered portion of the snap-fit rim 306. The snap-fit rim 306 is configured to slide over the tapered distal edge 318 and the outer surface of the poppet valve 310 when a force that matches or exceeds the set threshold is applied to the IV set. This is described in greater detail below with respect to FIG. 5.

The snap-fit rim 306 is not only removably coupled to the distal portion of the poppet valve 310 but is also removably coupled to the inlet port 308. In some embodiments, a proximal portion of the inlet port 308 extends (at least partially) into the housing 302 via the snap-fit rim 306, and a distal portion of the inlet port 308 protrudes out of the housing 302 and forms the distal portion of the delivery connector 300. The distal end of the inlet port 308 (and, thus, the distal portion of the delivery connector 300) is configured to couple with an IV line 22 (which corresponds to the IV tube 22 of the IV set 1 in FIG. 1).

In addition to extending into the housing 302, the proximal end of the inlet port 308 extends into the distal portion of the poppet valve 310. Specifically, an outer surface of the inlet port 308 is coupled to a distal portion of an inner surface of the fluid chamber 312. In some embodiments, the inlet port 308 is welded to the fluid chamber 312. In other embodiments, the inlet port 308 is ultrasonically welded to the fluid chamber 312 because ultrasonic welding limits the introduction of contaminants and degradation into the weld, produces devices with very narrow dimensional tolerances, and decreases production costs. Additionally, ultrasonic welding can be performed in clean rooms, which is beneficial when manufacturing medical devices.

The fluid chamber 312 occupies the distal portion of the poppet valve 310 and is configured to receive the fluid from the IV line 22 via the inlet port 308. The other side of the poppet valve 310—i.e., the proximal portion—is a coupling chamber 320. The coupling chamber 320 is positioned within the skirt 304 of the housing 302, and the wall of the coupling chamber 320 functions as a taper of a male luer formed by the proximal region of the delivery connector 300. When the male luer (i.e., the skirt 304 and the coupling chamber 320) is coupled to the luer 202, the coupling chamber 320 is positioned within the luer 202. The fluid chamber 312 is connected to the coupling chamber 320 via an outlet 314, and whether fluid can leave the fluid chamber 312 depends on whether the outlet 314 is sealed or exposed. That is, whether fluid can leave the fluid chamber 312 and flow out of the delivery connector 300 depends on whether the poppet valve 310 is in the sealed configuration (shown in FIG. 3) or the flow configuration (shown in FIG. 4).

In FIG. 3, the poppet valve 310 is in the sealed configuration because the plug 322 is blocking the outlet 314. The plug 322 can be made of a solid, polycarbonate material. Polycarbonate material is strong and touch, which makes the plug 322 more durable. Additionally, polycarbonate material can be made transparent, which is convenient for caregivers. Finally, polycarbonate material is easy to mold and thermoform, which is beneficial for manufacturing the plug 322, which has an unconventional shape, as described herein.

The plug 322 can include a distal post 324, a proximal post 330, and a sealing region 332 in between the distal post 324 and the proximal post 330. The distal post 324 is positioned within the fluid chamber 312, which occupies the space between the inlet port 308 and the outlet 314. In the sealed configuration, there is a gap 328 between the distal end of the distal post 324 and the inlet port 308. The gap 328 is between about 0.1 inches and about 0.4 inches. In some embodiments, the gap 328 is about 0.235 inches. The distal post 324 is coupled to or includes a biasing member 326 (e.g., a spring or an otherwise resilient component) that extends across the gap 328. Meaning, in some embodiments, the biasing member 326 is coupled to the distal end of the distal post 324. In other embodiments, the biasing member 326 is part of the distal post 324, such that the distal portion of the distal post 324 is positioned within the biasing member 326. In the embodiment shown in FIG. 3, the biasing member 326 is a compression spring, and the entirety of the distal post 324 is positioned within the spring 326, such that the spring 326 extends from the inlet port 308, over the gap 328 and the distal post 324, until the spring 326 reaches the sealing region 332.

In some embodiments of the present disclosure, the plug 322 can include a proximal post 330 that forms the proximal portion of the plug 322. When the plug 322 is in the sealed configuration, the proximal post 330 is positioned proximal to the outlet 314, extends through the coupling chamber 320, and protrudes beyond the proximal end of the skirt 304. In some embodiments, the length of the proximal post 330 that protrudes outside of the skirt 304 (and, thus, the poppet valve 310 and the housing 302) is the same as the length of the gap 328. Accordingly, the proximal post 330 can extend beyond the proximal end of the housing 302 by a length between about 0.1 inches and 0.4 inches. Optionally, 0.235 inches of the proximal post can protrude from the proximal side of the housing 302.

In some embodiments, the proximal post 330 has a diameter that is between about 0.05 inches and about 0.15 inches. The proximal post 330 can have a diameter of about 0.09 inches. Optionally, the proximal post 330 and the distal post 324 have the same diameter.

The proximal post 330 is configured to fluidly connect the fluid chamber 312 to the lumen 204 of the reception connector 200. This is discussed in greater detail below with respect to FIG. 4, the deformed configuration of the deformable valve 206, and the flow configuration of the poppet valve 310.

In between the distal post 324 and the proximal post 330 is the sealing region 332. When the poppet valve 310 is in the sealed configuration, the sealing region 332 fits within the outlet 314 to plug or seal the outlet 314. The sealing region 332 can be considered a poppet, and the surface of the outlet 314 can be considered a valve seat. In the embodiment shown, both the sealing region 332 and the outlet 314 are proximally tapered radially inward. Specifically, the sealing region 332 has a tapered surface 334, and the outlet 314 has a tapered surface 316. Both surfaces 334 and 316 have the same slope and form a seal when the sealing region 332 of the plug 322 is pushed into the outlet 314 by the spring 326. Because the outlet 314 is sealed by the plug 322, fluid from the IV set enters the fluid chamber 312 via the inlet port 308 and remains in the fluid chamber 312 until the outlet 314 is unsealed.

FIG. 4 illustrates a cross-sectional view of an anti-dislodgement connector assembly with the reception connector and the delivery connector coupled together, in accordance with aspects of the present disclosure. The reception connector 200 and the delivery connector 300 are coupled together by a luer lock formed by the luer 202, the skirt 304, and the coupling chamber 320. Particularly, the coupling chamber 320 is received by the luer 202, and flanges or lugs on an outer surface of the luer 202 engage with threads inside the skirt 304. As the reception connector 200 and the delivery connector 300 are coupled together, the plug 322 of the poppet valve 310 is advanced towards the deformable valve 206.

In response to the distal force applied by the deformable valve 206, the plug 322 moves distally towards the inlet port 308, which compresses the spring 326 and closes the gap (i.e., the gap 328 shown in FIG. 3) between the distal post 324 and the inlet port 308. In the embodiment shown, in which the spring 326 surrounds the entirety of the distal post 324, the spring 326 collapses on top of the distal post 324 as the plug 322 moves distally and closes the gap. Because the deformable valve 206 distally advances the plug 322 towards the inlet port 308, the sealing region 332 moves distally away from the outlet 314, and the poppet valve 310 enters the flow configuration. Once the gap is closed and the plug 322 can no longer move away from the deformable valve 206, the deformable valve 206 begins to compress and deform against the proximal post 330. In the deformed configuration, the deformable valve 206 buckles and moves away from the opening at the distal end of the luer 202, which means that the deformable valve 206 does not seal the luer 202.

When the poppet valve 310 is in the flow configuration and the deformable valve 206 is in the deformed configuration, fluid can flow from the IV line 22, through the connector assembly 100, and into the IV line 16. Specifically, fluid flows into the inlet port from an IV bag or an infusion pump via the IV line 20. The fluid then enters the fluid chamber 312 and flows through the outlet 314 into the coupling chamber 320 because the sealing region 332 has been distally advanced away from the outlet 314. The fluid then enters the lumen 204 because the deformable valve 206 is not sealing the opening of the reception connector 200. Finally, the fluid can exit the reception connector 200 through the outlet 208 and flow into the IV line 16 towards the patient.

As mentioned above, when the connector assembly 100 is assembled as shown in FIG. 4, the coupling chamber 320 is positioned within both the luer 202 and the skirt 304. When the poppet valve 310 is in the flow configuration, the coupling chamber 320 is fluidly connected to the fluid chamber 312. At the same time, when the deformable valve 206 is in the deformed configuration, the coupling chamber 320 is fluidly connected to the lumen 204. Accordingly, the coupling chamber 320 forms a fluid path between the inlet port 308, the fluid chamber 312, the lumen 204, and the outlet 208 when the reception connector 200 and the delivery connector 300 are coupled together. In particular, fluid can enter the assembly 100 through the inlet port 308, fill the fluid chamber 312 and surround the distal post 324, flow around the sealing region 332 and out the proximal outlet 314 towards the coupling chamber 320. In the coupling chamber 320, the fluid surrounds the proximal post 330 and flows into the lumen 204 of the reception connector 200, where it surrounds the deformable valve 206. This is a closed fluid path that reduces the occurrence of fluid leaking out of the connector assembly 100.

FIG. 5 illustrates a cross-sectional view of an anti-dislodgement connector assembly after the reception connector and the delivery connector have been decoupled by an outside force, in accordance with aspects of the present disclosure. If a patient tugs on the tubing of the IV set or a caregiver trips on the tubing, a force is applied to the IV set. As discussed above, if the force matches or exceeds a threshold, the force can decouple the reception connector 200 from the delivery connector 300. This decoupling of the connector assembly 100 is preferable to the IV set being dislodged from the patient, which subjects the patient to pain and, in some circumstances, infection. Additionally, when the reception connector 200 and the delivery connector 300 are disconnected, fluid flow through the IV set is interrupted, which reduces the occurrence of leaks and loss of fluid.

With reference to FIG. 5, when a sufficient force is applied to the IV set, the snap-fit rim 306 slides away from the inlet port 308 and over the tapered distal edge 318 of the poppet valve 310. A sufficient force will cause the snap-fit rim 306 to slide over the entirety of the poppet valve 310 until the housing 302 is disconnected from the poppet valve 310 and the inlet port 308. In some embodiments, the snap-fit rim 306 is configured to break away from the remainder of the housing 302 when a sufficient force is applied. Consequently, the snap-fit rim 306 remains coupled to the inlet port 308 and the poppet valve 310, and the remainder of the housing 302 is disconnected from the poppet valve 310, the inlet port 308, and the snap-fit rim 306.

The housing 302 remains coupled to the reception connector 200 via the luer lock connection formed by the luer 202, the skirt 304, and the coupling chamber 320 of the poppet valve 310. In some embodiments, the skirt 304 and the luer 202 can be threadably connected (i.e., screwed together), so the connection between the skirt 304 and the luer 202 is stronger than the connection between the snap-fit rim 306 and the poppet valve 310 and the inlet port 308. When the housing 302 separates from the remainder of the delivery connector 300, caregivers have a clear, visual indicator that the connector assembly 100 has been disconnected and that the IV set needs to be reset.

Furthermore, when the connector assembly 100 is decoupled, the deformable valve 206 is disconnected from the plug 322. As a result, the spring 326 proximally advances the plug 322 until the tapered surface 334 of the sealing region 332 is pressed up against the tapered surface 316 of the outlet 314, thereby forming a fluidic seal between the sealing region 332 and the outlet 314 and sealing the fluid chamber 312. Similarly, the deformable valve 206 expands back into its default configuration and seals the luer 202, which means that fluid can no longer flow in or out of the luer 202. Additionally, when the deformable valve 206 is in the default configuration, the outlet 208 is fluidly disconnected from the lumen 204.

The force required to decouple the reception connector 200 and the delivery connector 300 is between about three pounds and about five pounds. In some embodiments, the threshold force is about four pounds. The threshold force is significantly larger than the force required to deform the deformable valve 206 against the proximal post 330 of the plug 322. And the force required to distally advance the plug 322, compress the spring 326, and close the gap (i.e., gap 328 in FIG. 3). The force can be a sudden force (e.g., from a quick pull on the IV tubing) or a prolonged force (e.g., from an object or person leaning on the IV tubing).

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. An anti-dislodgement connector assembly configured to connect to an intravenous (IV) set and comprising a reception connector and a delivery connector, wherein the reception connector comprises a female luer and a deformable valve disposed within the female luer, and the delivery connector comprises: a housing comprising a proximal skirt and a snap-fit rim opposite the skirt; an inlet port coupled to the snap-fit rim and configured to transport fluid from the IV set to the delivery connector; a poppet valve coupled to the inlet port, proximally abutting the snap-fit rim, and comprising (a) a fluid chamber configured to receive the fluid from the inlet port and comprising a proximal outlet and (b) a plug comprising (i) a distal post positioned within the fluid chamber and coupled to the inlet port by a biasing member, (ii) a proximal post, and (iii) a sealing region positioned between the distal post and the proximal post and movable relative to the proximal outlet, wherein the proximal skirt and the poppet valve form a male luer configured to couple with the female luer of the reception connector, the poppet valve comprising a taper of the male luer, wherein, when the male luer is coupled with the female luer, (a) the poppet valve engages against the deformable valve such that the plug is distally displaced, thereby compressing the biasing member and forming a fluid passage between the sealing region and the proximal outlet and (b) the deformable valve deforms against the proximal post to fluidly couple the fluid chamber to the proximal outlet, wherein, when a force is applied to the IV set, the snap-fit rim slides away from the inlet port and over the poppet valve to decouple the housing from the inlet port and the poppet valve, and the biasing member proximally advances the sealing region into the proximal outlet to seal the fluid chamber.

Clause 2. The assembly of Clause 1, wherein the biasing member comprises a spring.

Clause 3. The assembly of Clause 1, wherein, when the male luer is not coupled with the female luer, there is a gap between the inlet port and the distal post, and the biasing member extends from the distal post and over the gap to contact the inlet port.

Clause 4. The assembly of Clause 3, wherein the distal post is at least partially positioned within an interior of the biasing member.

Clause 5. The assembly of Clause 1, wherein the force is at least about 4 pounds.

Clause 6. The assembly of Clause 1, wherein, when the force is applied to the IV set, the housing decouples from the inlet port and the poppet valve before the IV set is dislodged from a patient.

Clause 7. An anti-dislodgement connector configured to connect to an intravenous (IV) set, the connector comprising: a housing comprising a snap-fit rim and a proximal skirt configured to couple to a fluid reception device of the IV set; an inlet port coupled to the snap-fit rim and configured to receive fluid from the IV set; a poppet valve coupled to the inlet port, proximally abutting the snap-fit rim, and comprising (a) a fluid chamber configured to receive the fluid from the inlet port and connected to a surrounding environment by a proximal outlet and (b) a plug comprising (i) a distal post positioned within the fluid chamber and coupled to the inlet port by a biasing member and (ii) a proximal sealing region that is movable relative to the proximal outlet, wherein the proximal skirt and the poppet valve form a male luer and, when the male luer is coupled to the fluid reception device, the plug is distally advanced to compress the biasing member, distally displace the sealing region and fluidly couple the fluid chamber to the proximal outlet, wherein, when a force is applied to the IV set, the snap-fit rim slides away from the inlet port and over the poppet valve to decouple the housing from the inlet port and the poppet valve, and the biasing member proximally advances the sealing region into the proximal outlet to seal the fluid chamber.

Clause 8. The connector of Clause 7, wherein the snap-fit rim is positioned at a distal end of the housing and comprises a lip that protrudes radially inwards.

Clause 9. The connector of Clause 7, wherein the inlet port is welded to the poppet valve.

Clause 10. The connector of Clause 7, wherein a distal end of the poppet valve proximally abuts the snap-fit rim and comprises a tapered edge, the snap-fit rim being configured to slide up the tapered edge when the force is applied to the IV set.

Clause 11. The connector of Clause 7, wherein the distal post is at least partially positioned within an interior of the biasing member.

Clause 12. The connector of Clause 7, wherein the plug further comprises a proximal post protruding proximally from the sealing region, the proximal post being configured to engage against the fluid reception device when the connector is coupled to the fluid reception device.

Clause 13. The connector of Clause 7, wherein the proximal outlet and the sealing region are both proximally tapered radially inward, and, when the male luer is not coupled to the fluid reception device, the sealing region fits within the proximal outlet such that the sealing region seals the proximal outlet.

Clause 14. The connector of Clause 7, wherein the force is at least about 4 pounds.

Clause 15. An anti-dislodgement connector configured to connect to an intravenous (IV) set, the connector comprising: a housing with a snap-fit rim; an inlet port coupled to the snap-fit rim and configured to receive fluid from the IV set; a poppet valve positioned proximal to the snap-fit rim and within the housing, the poppet valve comprising (a) a fluid chamber configured to receive the fluid from the inlet port and connected to a surrounding environment by a proximal outlet and (b) a plug coupled to the inlet port by a biasing member and movable relative to the proximal outlet, wherein, when the connector is coupled to a fluid reception device of the IV set, the biasing member is compressed and the plug is distally displaced to expose the proximal outlet, thereby fluidly connecting the fluid chamber to the proximal outlet, wherein, when a force is applied to the IV set, the snap-fit rim slides away from the inlet port and over the poppet valve to decouple the housing from the inlet port and the poppet valve, and the biasing member proximally advances the plug into the proximal outlet to seal the fluid chamber.

Clause 16. The connector of Clause 15, wherein a distal end of the poppet valve proximally abuts the snap-fit rim and comprises a tapered edge, the snap-fit rim being configured to slide up the tapered edge when the force is applied to the IV set.

Clause 17. The connector of Clause 15, wherein the plug further comprises a post protruding proximally from the plug, the post being configured to engage the fluid reception device to form a fluid path from the fluid chamber and the proximal outlet to the fluid reception device.

Clause 18. The connector of Clause 15, wherein the proximal outlet and the plug are both proximally tapered radially inward, and, when the connector is not coupled to the fluid reception device, the plug fits within the proximal outlet such that the plug seals the proximal outlet.

Clause 19. The connector of Clause 15, wherein the force is at least about 4 pounds.

Clause 20. The connector of Clause 15, wherein a proximal portion of the housing and the poppet valve form a male luer at a proximal portion of the connector, with the proximal portion of the housing forming a skirt and the poppet valve forming a taper.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An anti-dislodgement connector assembly configured to connect to an intravenous (IV) set and comprising a reception connector and a delivery connector, wherein the reception connector comprises a female luer and a deformable valve disposed within the female luer, and the delivery connector comprises:
a housing comprising a proximal skirt and a snap-fit rim opposite the skirt;
an inlet port coupled to the snap-fit rim and configured to transport fluid from the IV set to the delivery connector; and
a poppet valve coupled to the inlet port, proximally abutting the snap-fit rim, and comprising (a) a fluid chamber configured to receive the fluid from the inlet port and comprising a proximal outlet and (b) a plug comprising (i) a distal post positioned within the fluid chamber and coupled to the inlet port by a biasing member, (ii) a proximal post, and (iii) a sealing region positioned between the distal post and the proximal post and movable relative to the proximal outlet,
wherein the proximal skirt and the poppet valve form a male luer configured to couple with the female luer of the reception connector, the poppet valve comprising a taper of the male luer,
wherein, when the male luer is coupled with the female luer, (a) the poppet valve engages against the deformable valve such that the plug is distally displaced, thereby compressing the biasing member and forming a fluid passage between the sealing region and the proximal outlet and (b) the deformable valve deforms against the proximal post to fluidly couple the fluid chamber to the proximal outlet, and
wherein, when a force is applied to the IV set, the snap-fit rim slides away from the inlet port and over the poppet valve to decouple the housing from the inlet port and the poppet valve, and the biasing member proximally advances the sealing region into the proximal outlet to seal the fluid chamber.

2. The assembly of claim 1, wherein the biasing member comprises a spring.

3. The assembly of claim 1, wherein, when the male luer is not coupled with the female luer, there is a gap between the inlet port and the distal post, and the biasing member extends from the distal post and over the gap to contact the inlet port.

4. The assembly of claim 3, wherein the distal post is at least partially positioned within an interior of the biasing member.

5. The assembly of claim 1, wherein the force is at least about 4 pounds.

6. The assembly of claim 1, wherein, when the force is applied to the IV set, the housing decouples from the inlet port and the poppet valve before the IV set is dislodged from a patient.

7. An anti-dislodgement connector configured to connect to an intravenous (IV) set, the connector comprising:
a housing comprising a snap-fit rim and a proximal skirt configured to couple to a fluid reception device of the IV set;
an inlet port coupled to the snap-fit rim and configured to receive fluid from the IV set; and
a poppet valve coupled to the inlet port, proximally abutting the snap-fit rim, and comprising a fluid chamber and a plug, the fluid chamber configured to receive the fluid from the inlet port and connected to a surrounding environment by a proximal outlet, and the a plug comprising a distal post, positioned within the fluid chamber and coupled to the inlet port by a biasing member, and a proximal sealing region that is movable relative to the proximal outlet,
wherein the proximal skirt and the poppet valve form a male luer and, when the male luer is coupled to the fluid reception device, the plug is distally advanced to compress the biasing member, distally displace the sealing region and fluidly couple the fluid chamber to the proximal outlet, and
wherein, when a force is applied to the IV set, the snap-fit rim slides away from the inlet port and over the poppet valve to decouple the housing from the inlet port and the poppet valve, and the biasing member proximally advances the sealing region into the proximal outlet to seal the fluid chamber.

8. The connector of claim 7, wherein the snap-fit rim is positioned at a distal end of the housing and comprises a lip that protrudes radially inwards.

9. The connector of claim 7, wherein the inlet port is welded to the poppet valve.

10. The connector of claim 7, wherein a distal end of the poppet valve proximally abuts the snap-fit rim and comprises a tapered edge, the snap-fit rim being configured to slide up the tapered edge when the force is applied to the IV set.

11. The connector of claim 7, wherein the distal post is at least partially positioned within an interior of the biasing member.

12. The connector of claim 7, wherein the plug further comprises a proximal post protruding proximally from the sealing region, the proximal post being configured to engage against the fluid reception device when the connector is coupled to the fluid reception device.

13. The connector of claim 7, wherein the proximal outlet and the sealing region are both proximally tapered radially inward, and, when the male luer is not coupled to the fluid reception device, the sealing region fits within the proximal outlet such that the sealing region seals the proximal outlet.

14. The connector of claim 7, wherein the force is at least about 4 pounds.

15. An anti-dislodgement connector configured to connect to an intravenous (IV) set, the connector comprising:
a housing with a snap-fit rim;
an inlet port coupled to the snap-fit rim and configured to receive fluid from the IV set; and
a poppet valve positioned proximal to the snap-fit rim and within the housing, the poppet valve comprising a fluid chamber and a plug, the fluid chamber configured to receive the fluid from the inlet port and connected to a surrounding environment by a proximal outlet, and the plug coupled to the inlet port by a biasing member and movable relative to the proximal outlet,
wherein, when the connector is coupled to a fluid reception device of the IV set, the biasing member is compressed and the plug is distally displaced to expose the proximal outlet, thereby fluidly connecting the fluid chamber to the proximal outlet, and
wherein, when a force is applied to the IV set, the snap-fit rim slides away from the inlet port and over the poppet valve to decouple the housing from the inlet port and the poppet valve, and the biasing member proximally advances the plug into the proximal outlet to seal the fluid chamber.

16. The connector of claim 15, wherein a distal end of the poppet valve proximally abuts the snap-fit rim and comprises a tapered edge, the snap-fit rim being configured to slide up the tapered edge when the force is applied to the IV set.

17. The connector of claim 15, wherein the plug further comprises a post protruding proximally from the plug, the post being configured to engage the fluid reception device to form a fluid path from the fluid chamber and the proximal outlet to the fluid reception device.

18. The connector of claim 15, wherein the proximal outlet and the plug are both proximally tapered radially inward, and, when the connector is not coupled to the fluid reception device, the plug fits within the proximal outlet such that the plug seals the proximal outlet.

19. The connector of claim 15, wherein the force is at least about 4 pounds.

20. The connector of claim 15, wherein a proximal portion of the housing and the poppet valve form a male luer at a proximal portion of the connector, with the proximal portion of the housing forming a skirt and the poppet valve forming a taper.
